(19) United States
(12) Patent Application Publication
(10) Pub. No.: US 2012/0108972 A1
(43) Pub. Date: May 3, 2012

(54) ULTRASOUND DIAGNOSTIC APPARATUS AND METHOD FOR TRACING MOVEMENT OF TISSUE

(76) Inventors: Koji Miyama, Tokyo (JP); Masafumi Ogasawara, Tokyo (JP)

(21) Appl. No.: 13/283,460

(22) Filed: Oct. 27, 2011

(30) Foreign Application Priority Data
Oct. 27, 2010 (JP) 2010-241315

(51) Int. Cl. A61B 8/08

(52) U.S. Cl. 600/443

(57) ABSTRACT

An ultrasound diagnostic apparatus including a transmitting and receiving unit that transmits an ultrasound wave to a target object and receives the ultrasound wave as ultrasound data reflected from a certain region of the target object including a blood vessel, an image generation unit that generates an ultrasound image as a sectional image of the certain region, and a region of interest setting unit that sets a region of interest including a plurality of divided regions in the ultrasound image at a designated time. The region of interest is generated from the stored ultrasound data. The apparatus further includes a tracing unit that traces movement of tissue in the target object corresponding to the divided regions from the designated time to sequentially following thereafter, and a movement measuring unit that measures a movement distance of the tissue at a predetermined time based on the traced movement of the tissue.

### CROSS REFERENCE TO RELATED APPLICATIONS

This application claims the benefit of Japanese Patent Application No. 2010-241315 filed Oct. 27, 2010, which is hereby incorporated by reference in its entirety.

### BACKGROUND OF THE INVENTION

The embodiments described herein relate to an ultrasound diagnostic apparatus for diagnosing blood vessel using ultrasound.

In recent years, the number of patients diagnosed with a circulatory condition, such as cerebral infarction and cardiac infarction, are on the rise. To prevent such disease, it is important to detect a symptom of arteriosclerosis in its early stage and to improve the patient's lifestyle.

To detect such symptom of arteriosclerosis in its early stage, Japanese unexamined publication 2002-238903A (hereinafter “JP '903”) discloses an ultrasound diagnostic apparatus. An operator sets a mark for tracing on a surface of a plaque in a B (brightness)-mode image displayed in a monitor of the ultrasound diagnostic apparatus. And the ultrasound diagnostic apparatus traces a diameter of a blood vessel and a blood vessel wall by calculating a correlation of the brightness of pixels in a region of interest, including the previously set mark for tracing. Japanese unexamined publication 2010-110373A (hereinafter “JP '373”) discloses an ultrasound diagnostic apparatus for tracing a blood vessel wall of a surface of a plaque in a B-mode display by using pattern matching method.

Unfortunately, the brightness of the image value as described in JP '903 may alter the diameter of the blood vessel or the blood vessel wall depending on the image data processing. Also, JP '903 and JP '373 trace the surface of the inner wall of the blood vessel wall. For example, to understand the characteristic of the plaque in the blood vessel, it is important to trace inside of the plaque as well as the surface of plaque. Generally, the factors inducing to plaque rupture are the size of a fat core and a thickness of the fiber membrane covering the fat core. Thus, even when the plaque surface is not being moved so much, the size of the fat core or the thickness of the fiber membrane can be estimated by monitoring whether the inside of the plaque is largely moving. Therefore, it is important to understand the movement inside the plaque to understand the characteristic of plaque.

It is desirable that the problems described previously are solved.

### BRIEF DESCRIPTION OF THE INVENTION

A first aspect of an ultrasound diagnostic apparatus includes a transmitting and receiving unit for transmitting an ultrasound to a target object in sequence and for receiving the ultrasound as ultrasound data reflected from a certain region of the target object including a blood vessel in sequence. A memory unit stores the received ultrasound data in sequence. An image generation unit generates an ultrasound image as a sectional image of the certain region based on the received ultrasound data, and a display unit displays the ultrasound image generated by the image generation unit. The ultrasound diagnostic apparatus further includes a region of interest setting unit for setting a region of interest having a plurality of divided regions in an interest part of the ultrasound image displayed in the display unit at designated time. The region of interest is generated by ultrasound data stored in the memory unit. A tracing unit traces movement of tissue in the target object corresponding to the plurality of divided regions of the region of interest set for the ultrasound image at the designated time and sequentially following thereafter. A movement measuring unit measures the movement distance of the tissue at the predetermined time based on the movement of tissue traced by the tracing unit.

In the second aspect, the region of interest setting unit sets the region of interest as a whole in square divided regions each of which is square, and the squares are aligned vertical and horizontal directions.

In the third aspect, the region of interest setting unit can change the size of the divided regions of the region of interest to a designated size.

In the fourth aspect, the region of interest setting unit sets the region of interest as a whole in circular-shaped, elliptically shaped, fan-shaped or toric-shaped divided regions each of which is fan-shaped and aligned in radiated and circular directions.

In the fifth aspect of the ultrasound diagnostic apparatus, the tracing unit traces the movement of the tissue in the target object by using an optical flow method between the ultrasound images.

In the sixth aspect, the optical flow method includes a gradient using a spatial brightness gradient.

In the seventh aspect of the ultrasound diagnostic apparatus, the tracing unit traces determines that all of the regions of interest are moved and displays the moved regions of interest in the display unit when the amount of movement of each divided region is identical and moving to the identical direction.

In the eighth aspect of the ultrasound diagnostic apparatus, the interest part of the region of interest includes a plaque formed on an inner wall of the blood vessel.

According to the ultrasound diagnostic apparatus, it is possible to trace tissue inside of a blood vessel wall by setting the region of interest having a plurality of divided regions on the interest part and tracing the movement of tissue in the target object that corresponds to each region of the plurality of divided regions.

Further objects and advantages of the present invention will be apparent from the following description of the preferred embodiments of the invention as illustrated in the accompanying drawings.

### BRIEF DESCRIPTION OF THE DRAWINGS

FIG. 1 is an overall diagram of ultrasound diagnostic apparatus.

FIG. 2 is a flowchart showing an exemplary method of measuring blood vessel.

FIG. 3 is a diagram explaining the brightness gradient of the grayscale image.

FIG. 4 is a diagram of setting the region of interest (ROI) to the long axis direction of a blood vessel by an operator.

FIG. 5 is a diagram of setting the region of interest (ROI) to the short axis direction of a blood vessel by an operator.

FIG. 6 is another diagram of setting the ROI to the short axis direction of a blood vessel by an operator.

FIG. 7(a) is a diagram showing a set of ultrasound images displayed by a display unit.

FIG. 7(b) is a vector diagram showing the movement of a plurality of divided regions (DR) moved between a predetermined time T1 and a predetermined time T2.

FIG. 8 is an example of displaying the graph of a traced result sorted by the ultrasound image.

FIG. 9 is an example of displaying the graph of a traced result sorted by the ultrasound image.

### DETAILED DESCRIPTION OF THE INVENTION

### Configuration of the Ultrasound Diagnostic Apparatus 100

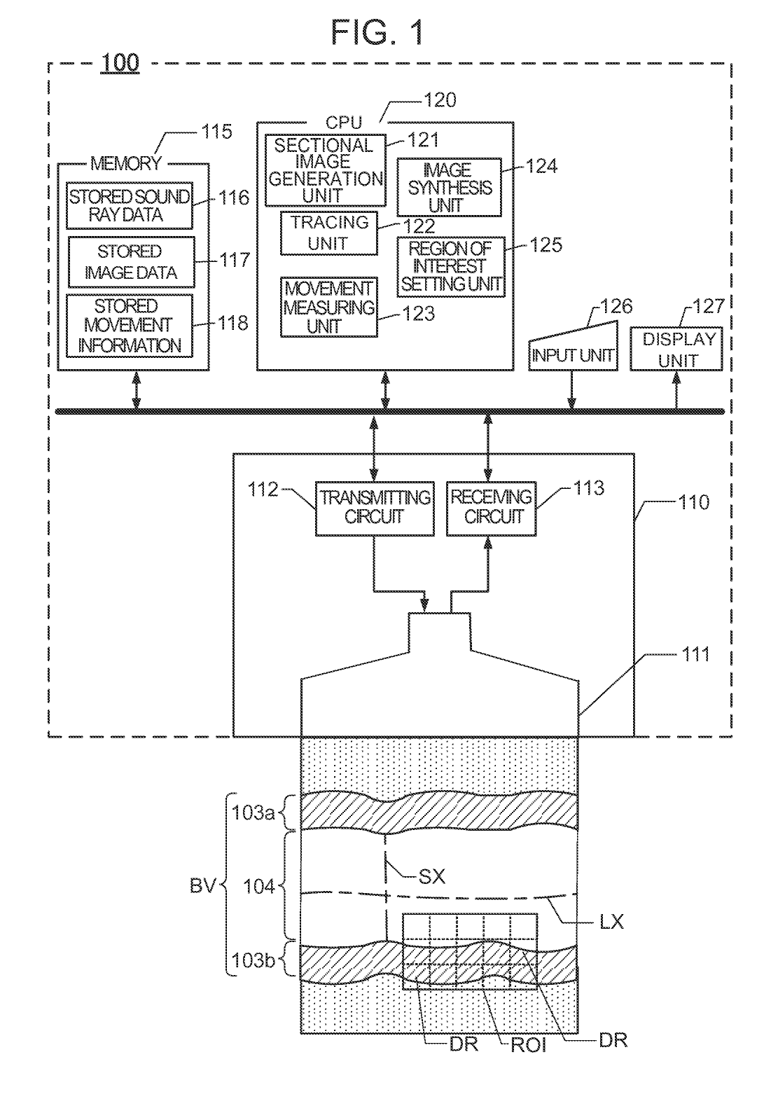

FIG. 1 is a block diagram showing an exemplary configuration of the ultrasound diagnostic apparatus 100. The ultrasound diagnostic apparatus 100 includes a transmitting and receiving unit 110 connected to a parallel bus, a memory 115, a CPU (central processing unit) 120, an input unit 126 for inputting through a mouse or a keyboard, and a display unit 127 for LCD unit.

The transmitting and receiving unit 110 includes an ultrasound probe 111, a transmission circuit 112, and a receiving circuit 113. The ultrasound probe 111 includes a plurality of ultrasound transducers, including a 1-dimensional or a 2-dimensional transducer array. The ultrasound transducers transmit an ultrasound based on a driving signal applied to a target object, receive ultrasound echoes reflected from the target object, and output a receiving signal.

The transmission circuit 112 includes a plurality of channels and generates a plurality of driving signals applied from the plurality of ultrasound transducers. The transmitting circuit 112 can adjust an amount of delay in the plurality of driving signals so that the ultrasound transmitted from the plurality of ultrasound transducers forms an ultrasound beam thereafter. Also, the transmitting circuit 112 can provide to the ultrasound probe 111 a plurality of driving signals set for transmitting an ultrasound transmitted from the plurality of ultrasound transducers all at once to the image region of the target object.

The receiving circuit 113 has a plurality of channels, amplifies a plurality of analog receiving signals outputted from each transducer of the plurality of ultrasound transducers, and converts to digital receiving signals. Moreover, based on a received delay pattern selected from the transmitting and receiving unit 110, the receiving circuit 113 applies each delay time to the plurality of receiving signals and processes receiving focus by adding all of the receiving signals. Due to the receiving focus processing, the sound ray data with focused ultrasound echo is formed.

In this embodiment, the ultrasound probe 111 transmits ultrasound from the surface of the target object to a blood vessel BV inside the target object. Also, the ultrasound probe 111 receives an ultrasound echo from the target object, including blood vessel. The transmitting and receiving unit 110 repeats the transmission of the ultrasound and reception of the ultrasound echo for outputting the sound ray data in sequence. The sound ray data processes logarithmic compression, gain adjustment or low-pass filter processing in the receiving circuit 113, and processes an attenuation correction in accordance to a depth of the reflecting position of ultrasound. The processed sound ray data is sequentially stored in the memory 115 through the parallel bus.

The memory 115 includes capacity for storing a plurality of frames of the sound ray data 116 or sectional image data 117 generated by an image generation unit 121.

CPU 120 includes the image generation unit 121, the tracing unit 122, the movement measuring unit 123, the image synthesis unit 124, and the region of interest setting unit 125.

The image generation unit 121 includes an image data generation function for generating sectional image data of B-mode by inputting the supplied sound ray data. The image generation unit 121 converts the B-mode sectional image data into the sectional image data that complies with the scanning system of a normal television signal, performs image processing necessary for a gradation process, transmits to image synthesis unit 124 or display unit 127, and sequentially stores into memory 115.

Also, in live mode, the image generation unit 121 converts the directly supplied sound ray data into the sectional image data in accordance to a scanning method. In freeze mode, the image generation unit 121 converts the sectional image data 117 stored in the memory 115 into the sectional image data in accordance to the scanning method. Moreover, during freeze mode, if the memory 115 stores the sound ray data 116 instead of the sectional image data 117, the image generation unit 121 generates the B-mode sectional image data.

For the region of interest setting unit 125, an operator sets the region of interest (ROI) in the ultrasound image using the input unit 126, such as a mouse. The region of interest setting unit 125 extracts the image data for the ROI. Once the ROI is set, the region of interest setting unit 125 extracts the sectional image data of the ROI for the sectional image data 117 (or, the sound ray data 116 stored in memory 115) that is stored in the memory 115. The sectional image data extracted from the ROI set by the region of interest setting unit 125 are supplied to the tracing unit 122. The ROI includes a plurality of divided regions (DR) as described at the bottom of FIG. 1.

The tracing unit 122 traces a divided region (DR) of the region of interest (ROI) that is moving in a vector direction from a designated time. To trace the divided region DR of the ROI, a method of calculating the velocity field of the motion of the object in the moving image (optical flow) is used. There are many methods of calculating an optical flow. In one embodiment, a gradient method is suitable for tracing a blood vessel wall. More specifically, the gradient method was suitable for tracing fine movements. The result of the tracing unit 122 tracing each divided region DR of the ROI is transmitted to the image synthesis unit 124, the movement measuring unit 123, and the memory 115. Also, when the tracing unit 122 determines that the blood vessel is moving as a whole and transmits such signal to the display unit 127, the display unit 127 can display the ROI in accordance with the movement of the blood vessel.

The movement measuring unit 123 measures the distance of movement in tissue for a predetermined duration based on the movement of the divided region DR in the ROI in which the tracing unit 122 traced. The traced result measured by the movement measuring unit 123 is transmitted to the image synthesis unit 124, the memory 125, and the display unit 127. The traced result transmitted to the memory 115 is stored as the movement information 118. The traced result transmitted to the display unit 127 is displayed as a movement of tissue inside the divided region DR in the ROI in real-time.

The image synthesis unit 124 synthesizes the sectional image data supplied from the image generation unit 121, the movement information 118 traced by the tracing unit 122 and the traced result measured in the movement measurement unit 123, and synthesizes two images therewith. Image synthesis unit 124 can retrieve the sound ray data 116 or sectional image data 117 stored in the memory 115 on a necessary basis.

A diagram of a blood vessel in the long axis direction inside the target object in FIG. 1 is explained below.

A blood vessel includes a blood vessel wall 103, which is surrounding a blood flow region 104. Blood vessel wall 103 includes a front wall 103a, which is a wall closer to the ultrasound probe 111, and a back wall 103b, which is a wall farther from the ultrasound probe 111. In FIG. 1, the region of interest (ROI) set by the region of interest setting unit 125 is positioned in the back wall 103b. The long axis direction LX refers to the blood vessel extending in the longitudinal direction from the center of the blood flow region 104, and the short axis direction SX refers to the cross-section of the blood vessel which is in a vertical straight line direction to the long axis direction LX.

<Method for Measuring a Blood Vessel>

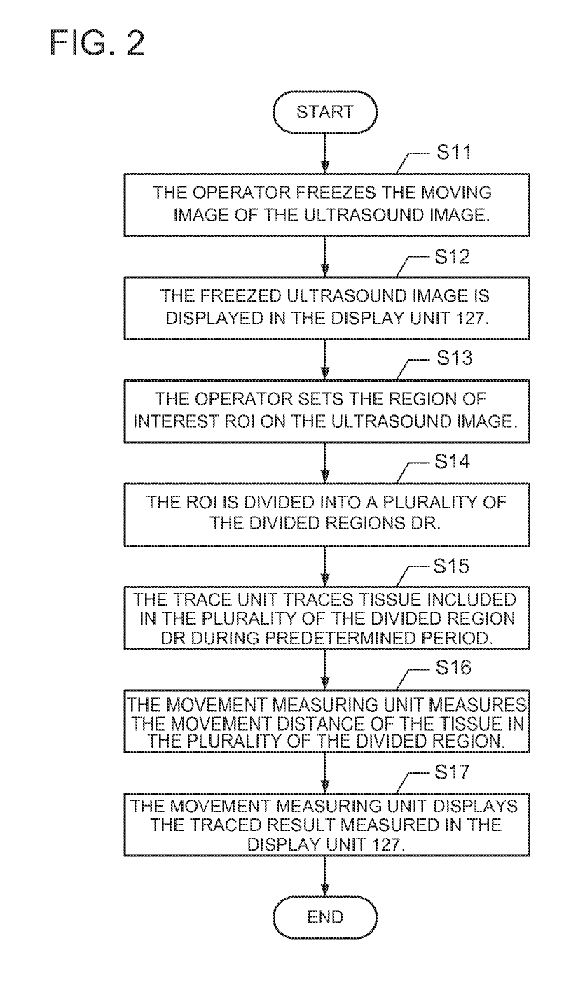

FIG. 2 is a flow chart showing an exemplary method for measuring the blood vessel. In step S11, the operator confirms that the moving image of the ultrasound image is stably obtained, and presses the freeze button. In step S12, when the freeze button is pressed, the sound ray data 116 or the sectional image data 117 acquired during a few seconds after the freeze button is pressed is stored in the memory 115, and the ultrasound image stored in the first frame is displayed by the display unit 127. The sound ray data 116 or the sectional image data 117 acquired a few seconds after the freeze button is pressed can be stored in the memory 115.

In step S13, the operator sets the ROI in the first frame of the ultrasound image displayed by the display unit 127 using the input unit 126, such as a mouse, connected by parallel bus. The operator can easily set the ROI as a blood vessel inside the target object displayed by the display unit 127 using the region of interest setting unit 125. The region of interest should be set to surround the region of interest. The region of interest should be set as a square.

In step S14, the ROI is divided into a plurality of divided regions DR.

The region of interest setting unit 125 can set a plurality of divided regions automatically depending on the size of the set ROI. Also, the operator can set the ROI into an arbitrary number of divided regions DR using the region of interest setting unit 125.

In step S15, the trace unit 122 traces the movement of tissue included in a divided region DR in the ROI using frames from the first frame of the ultrasound image to the frame after the predetermined time of the ultrasound image. The optical flow method is used to trace the region of interest.

In step S16, the movement measuring unit 123 measures the movement distance of the divided region DR of the ROI.

In step S17, the display unit 127 displays the traced result measured by the movement measuring unit 123. The measured traced result is displayed as a vector for each of the divided regions DR, or displayed as a graph including time as an axis. A graph can be displayed next to the displayed ultrasound image or on a separate window.

<Tracing the ROI by the Gradient Method>

An optical flow method used by the tracing unit 122 for tracing the movement of tissue in each divided region DR of the ROI, at step S15, is explained below.

The optical flow method includes a characteristic matching method, which is a method for matching the characteristic of images and calculating the movement, and includes a gradient method, which is a method for calculating the movement by calculating the gradient of the contrasting density (brightness) of an image for comparing the contrasting density of the image. In a particular embodiment, both the characteristic matching method and the gradient method are applied to the ultrasound image including the blood vessel displayed in the B-mode. As a result, less difference in tracing was found in the gradient method.

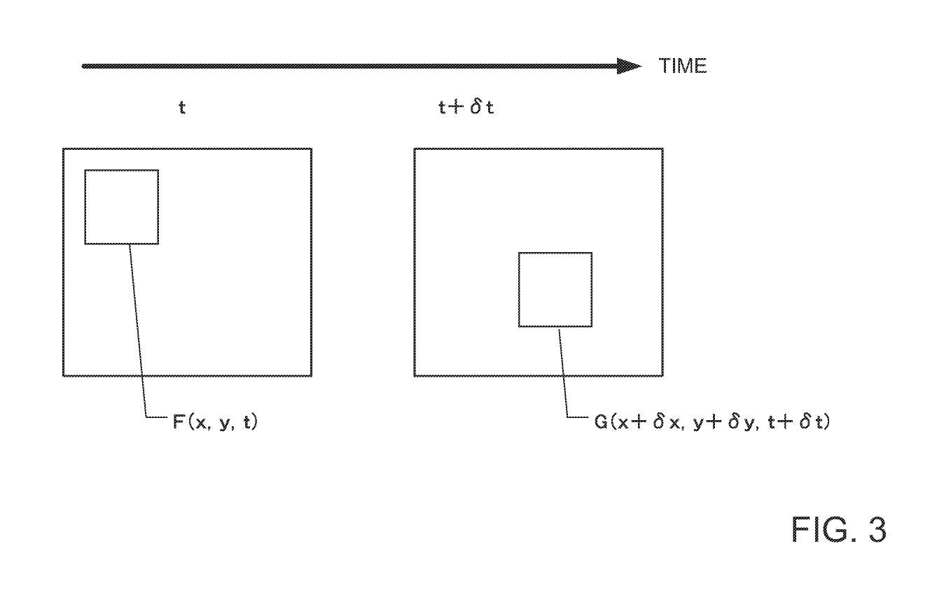

In the gradient method, the contrasting density image F (p, t) includes a gradient of contrasting density (brightness gradient), as shown in FIG. 3. Using the gradient of the contrasting density, a movement of tissue included in the ROI is traced.

As shown in FIG. 3, a contrasting density image “F” at time “t” (p, t) moved with even contrasting density after a minute duration (δ, t) is calculated as a contrasting density image G (p+δp, t+δt). The movement distance is calculated using the following equation:

$${h}_{0} = 0 , \\ {h}_{k + 1} = {h}_{k} + \frac{Σ w ( p ) {F}^{′} ( p + {h}_{k} ) [ G ( p ) - F ( p + {h}_{k} ) ]}{Σ w ( p ) {{F}^{′} ( p + {h}_{k} )}^{2}} \qquad equation 1$$

The movement distance (vector) of tissue in the ROI is calculated iteratively using equation 1, where “h” represents the distance of approximate movement, w (p) represents the weight coefficient, F (p) represents the contrasting density image before movement, and G (p) represents the contrasting density image after movement. F′ (p) represents the first derivation. The gradient method is efficient for tracing minute movements, such as movement of a blood vessel wall due to the heartbeat.

As explained above, minute movement of the blood vessel wall due to the heartbeat can be accurately traced by tracing the movement of tissue included in each divided region DR of the ROI using the gradient method.

<Setting the ROI>

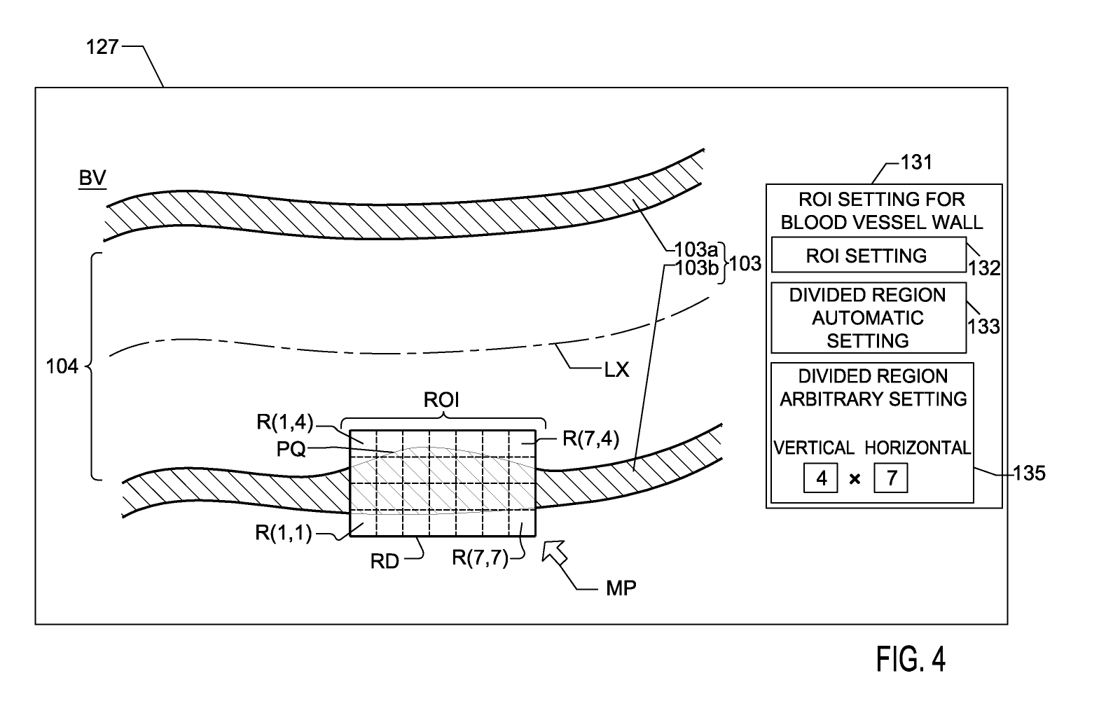

FIG. 4 shows one example of the ROI on the blood vessel extending in the long axis direction, which was set by an operator, as displayed by the display unit 127. In step S13 of FIG. 2, the ROI is set, and in step S14, the ROI is divided into a plurality of divided regions DR.

An operator checks the ultrasound image of the first frame displayed by the display unit 127. Then, an operator checks whether the blood vessel extending in the long axis direction is a sectional image that can set the ROI. If the sectional image is an image that can set the ROI, an operator clicks the ROI setting button (not described on figure) using the input unit 126, such as the mouse pointer MP. The region of interest setting unit 125 (refer to FIG. 1) displays the ROI setting window 131 for the blood vessel wall on the display unit 127.

The ROI setting window 131 for the blood vessel wall includes the ROI setting button 132 for setting the region of interest, the divided region automatic setting button 133, and the divided region arbitrary setting button 135.

The ROI setting button 132 is clicked using the mouse pointer MP by the operator, and the ROI shown in the bold line on the outer frame is created by dragging with the mouse pointer MP. At this point, the ROI shown in FIG. 4 is not yet created, and only the outer frame is displayed. The determined ROI will be a region for tracing by the tracing unit 122. Moreover, plaque PQ is located on the back wall 103b of the blood vessel wall 103, and the operator sets the ROI on the back wall 103b, including plaque PQ, as an interest part.

Then, either the divided region automatic setting button 133 or the divided region arbitrary setting button 135 is clicked using the mouse pointer MP by an operator. When the divided region automatic setting button 133 is clicked by the mouse pointer, the ROI is divided into predetermined sized divided regions. When the divided region arbitrary setting button 135 is clicked after the numbers of divisions on both vertical and horizontal directions are inputted by the operator, the ROI is divided into the inputted number of divided regions. In FIG. 4, four vertical divisions and seven horizontal divisions are inputted, and the region of interest is divided into 28 divided regions, which is displayed by the displaying unit 127. In the explanation below, a specific divided region DR of the divided regions DR is displayed as “R” (m, n) as necessary.

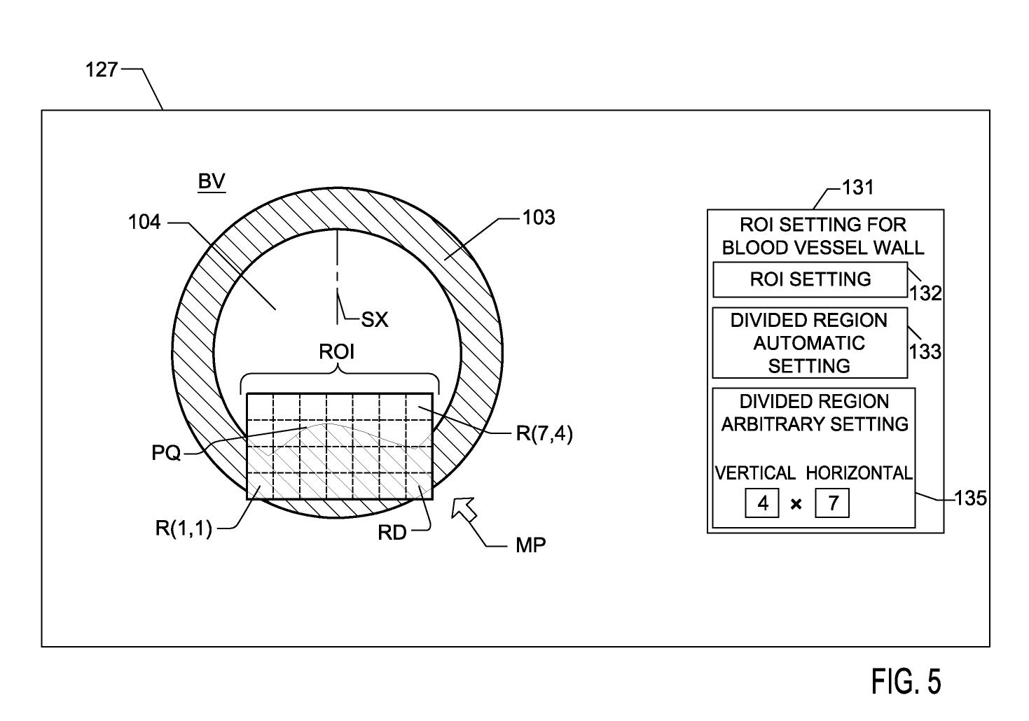

In FIG. 5, one example of the ROI in the short axis direction SX of the blood vessel BV, which was set by an operator, is displayed by the display unit 127. In step S13 in FIG. 2, the ROI is set, and in step S14, the ROI is divided into a plurality of divided regions DR.

As similar to the blood vessel BV extending in the long axis direction shown in FIG. 4, the ROI surrounds the plaque PQ on the blood vessel wall 103. Also, in FIG. 5, the ROI is divided into four vertical divisions and seven horizontal divisions, and 28 divided regions are displayed by the display unit 127.

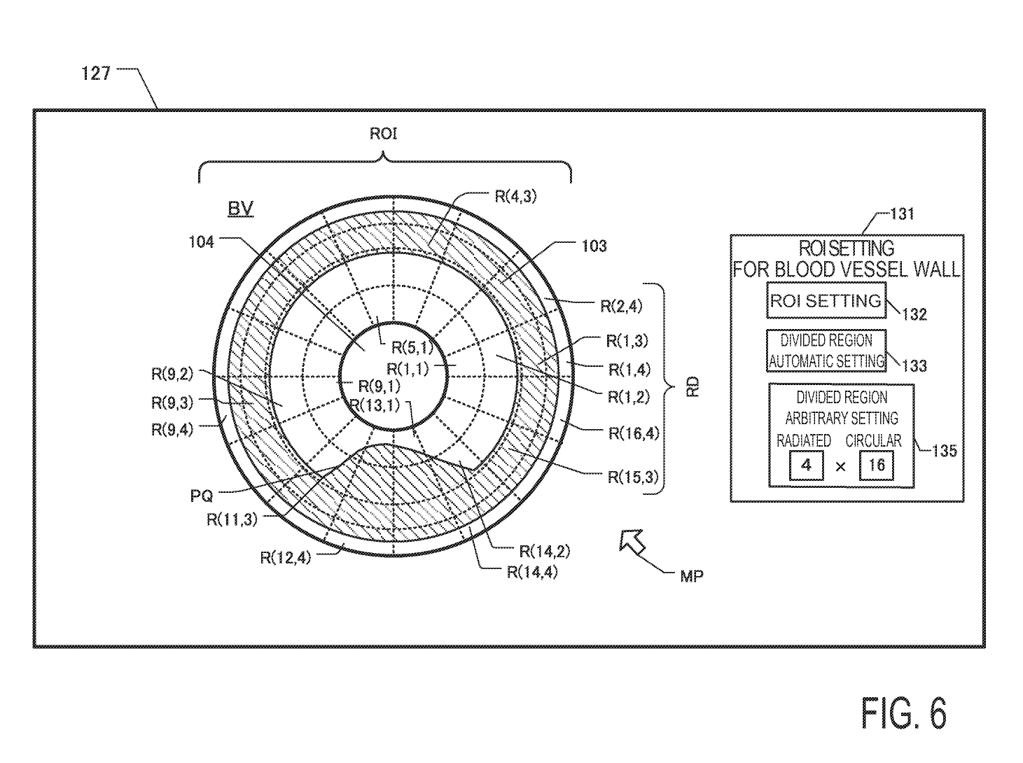

In FIG. 6, another example of the ROI in the short axis direction SX of the blood vessel BV, which was set by an operator, is displayed by the display unit 127. Normally, the blood vessel in the short axis direction SX is toric shape, and in some cases, it is preferred to set the region of interest in a toric shape. In FIG. 6, the operator clicks the ROI setting button using the mouse pointer MP, and the ROI, displayed with the toric-shaped frame in bold line, is created by dragging with the mouse pointer MP. At this point, the divided region DR as shown in FIG. 6 is not drawn yet, and only the toric-shaped frame is displayed. This specified ROI is a region to be traced by the tracing unit 122.

Then, either the divided region automatic setting button 133 or the divided region arbitrary setting button 135 is clicked using the mouse pointer MP. When the divided region automatic setting button 133 is clicked by the mouse pointer, the ROI is divided into predetermined sized divided regions. As different from the divided regions DR in FIG. 4 and FIG. 5, FIG. 6 shows a fan-shaped divided region DR. Also, when the divided region arbitrary setting button 135 is clicked after the dividing numbers are inputted into the radiated direction and circular direction, the divided regions DR are divided into the number of divisions inputted into the ROI. In FIG. 6, four divisions in the radiated direction and sixteen divisions in the circular directions are inputted, and the ROI that is displayed by the display unit 127 is divided into 64 divided regions. For example, if the toric-shaped region of interest is considered to be in the center, R(1,1) R(1,2) R(1,3) R(1,4) are set in the divided regions, starting at 0 degrees from the inside to the outside, and R(9,1), R(9,2), R(9,3), R(9,4) are set on the opposite points. The tracing unit 122 measures the movement distance of tissue at a predetermined time for these divided regions DR.

Moreover, to almost equate the area of each of the divided regions DR, the interval between radiated directions become smaller in an outward direction. FIG. 6 shows the example of tracing unit 122 tracing the movement of tissue in the divided regions DR. Since, in FIG. 6, the divided regions DR including the plaque PQ are R(11,1)-(11,4) to R(14,1)-R(14,4), the tracing unit 122 can trace the movement of tissue only in these sixteen divided regions. For example, the select button for selecting the region for tracing with the tracing unit 122 can be set in the ROI setting window 131, and the divided region DR necessary for the operator can be selected using the mouse pointer MP. On the other side, the operator can select unnecessary divided regions DR using the mouse pointer MP.

FIG. 6 shows the toric-shaped ROI. However, the ROI can be circular-shaped or elliptically shaped. Also, if the ROI is fan-shaped, only the region where the plaque PQ is present in FIG. 6 can be set as the region of interest.

<Tracing Information of the ROI>

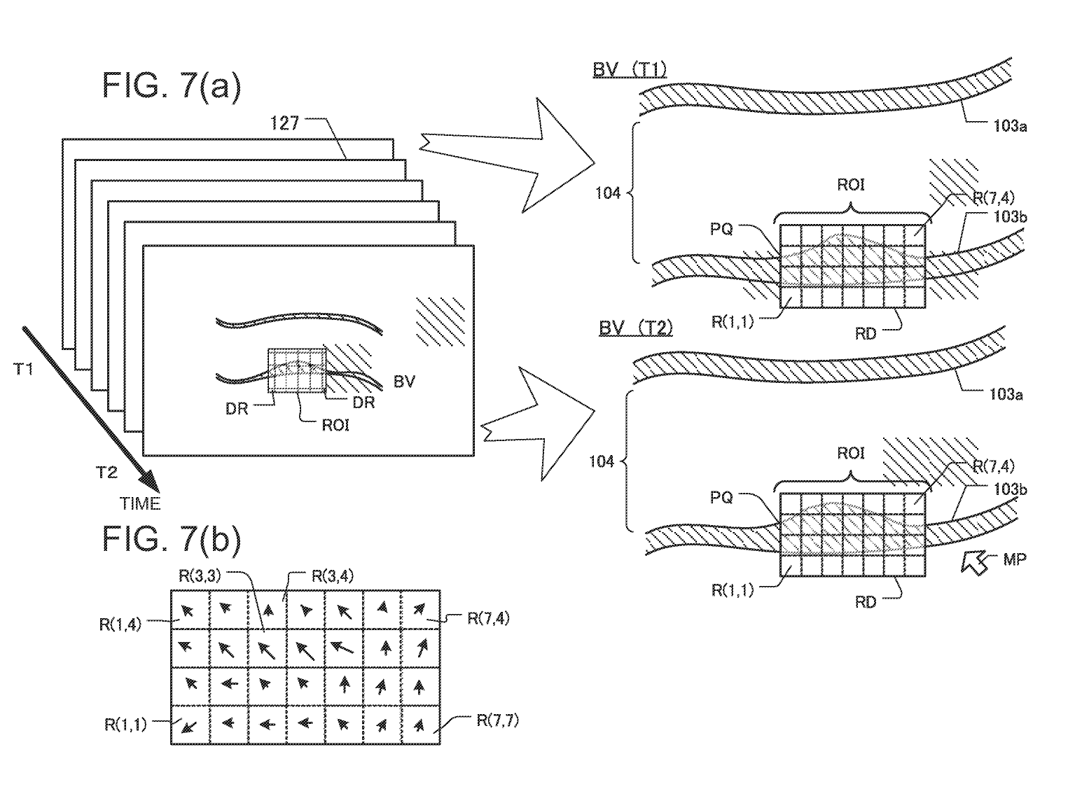

FIG. 7(a) shows a sequence of the ultrasound image displayed by the display unit 127 after setting the ROI. The left side of FIG. 7(a) shows a plurality of frames of the ultrasound moving image acquired from the predetermined time T1 to the predetermined time T2, which refers to a time after the predetermined time, being played, and the right side of FIG. 7(a) are abstracts of the ultrasound image of a T1 frame and ultrasound image of a T2 frame.

FIG. 7(b) is a vector diagram showing the maximum movement of the divided region DR in the ROI from the predetermined time T1 to time T2. The traced result measured in the movement measuring unit 123 is displayed in the display unit 127.

Due to the heartbeat, the sectional plane shape in the long axis direction of the blood vessel from time T1 to time T2 changes, so as the shape of the plaque PQ changes. Accordingly, tissue in a plurality of divided regions R(1,1)-R(7,4) in the ROI moves in the horizontal direction or the vertical direction of the display. In this embodiment, twenty-eight divided regions are set, and in each divided region DR, the amount of movement from time T1 to time T2 is measured by the movement measuring unit 123 (refer to FIG. 1).

The traced result measured by the movement measuring unit 123 can be displayed as a vector in each divided region as shown in FIG. 7(b). The direction of the arrow is the direction of the movement of tissue, and the length of the arrow is the maximum movement from time T1 to time T2. For example, the divided region R(3,4) has a short vector length (size), thus the maximum movement of tissue in the divided region R (3,4) is understood to be small. On the other hand, the divided region R (3,3) has a long vector length, thus the maximum movement of the tissue in the divided region R(3,3) is understood to be large. The divided region R(3,4) corresponds to the surface of the plaque PQ in FIG. 7(a), and the divided region R(3,3) corresponds to the inside of the plaque PQ in FIG. 7(a). As a result, the maximum movement of the plaque on the blood wall 103 displayed in the ultrasound image has small movement on the surface, but has a large movement inside. Therefore, the operator is able to accurately determine the characteristic of the plaque, better than the result acquired only by tracing the surface of the plaque.

The traced result measured by the movement measuring unit 123 can be displayed in color, such as displaying a vector larger than a first threshold value in orange, and a second threshold value that is larger than the first threshold value in red.

For example, all of the blood vessel BV might be moved when the contact between the ultrasound probe 111 and the target object is out of alignment. Therefore, the tracing unit 122 determines that, when the amount of movement of each divided region DR is almost identical and each divided region DR moves in the identical direction, tissue in each divided region DR is not determined to be moving, rather the tracing unit 122 determines that all of the blood vessel is moving. In such a case, the ROI indicated in FIG. 7(a) displays the movement starting from initially indicated or set position to following the position thereof. The movement measuring unit 123 displays the vector of each divided region DR as indicated in FIG. 7(b) using the amount of movement subtracted from the total amount of movement. The total amount of movement is calculated by averaging the amount of movement between divided regions R(1,1)-R(7,4).

<Example 1 of Displaying the Traced Result>

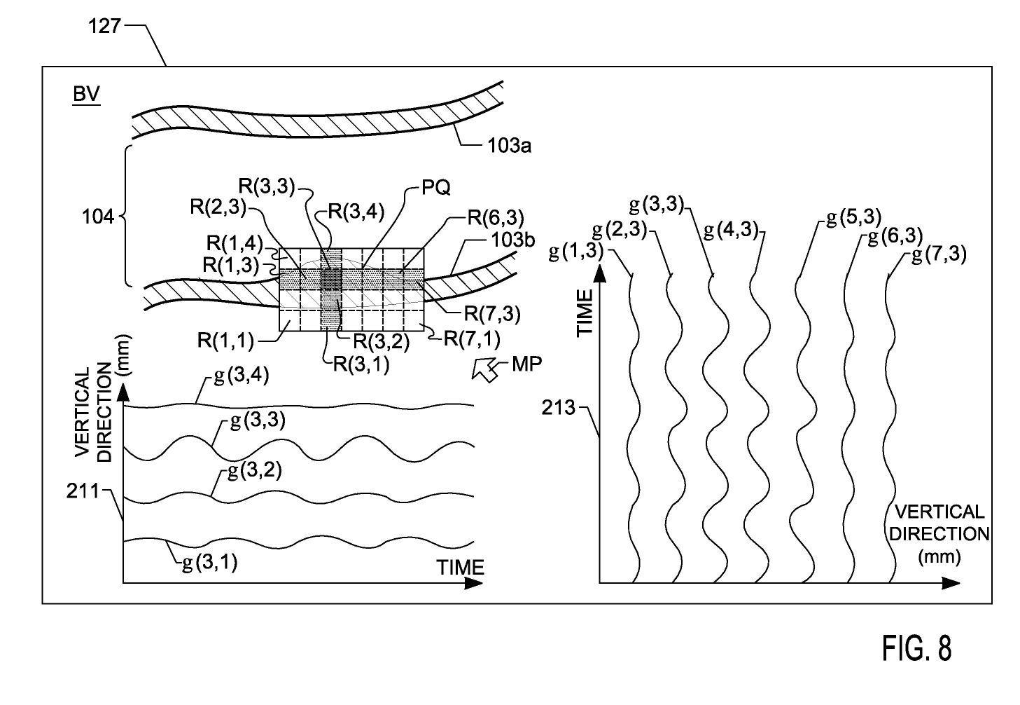

FIG. 8 shows the first example of the lined up graph of the traced result measured by the movement measurement unit 123 and the ultrasound image, for displaying on the display unit 127. These graphs are displayed based on the total movement of 28 divided regions R(1,1)-R(4,7) indicated in the region of interest as shown in FIG. 7(a).

On top left of the FIG. 8, the ultrasound image of the blood vessel wall in the long axis direction is displayed. On the ultrasound image, divided regions DR of the ROI set by an operator are sequentially displayed. At this point, among the divided regions DR, when the operator wants to observe one row of the divided regions R(3,1), R(3,2) R(3,3) and R(3,4), the operator selects the divided region R(3,1), R(3,2) R(3,3) and R(3,4) using the mouse pointer MP. One row of the selected DR is displayed in a different color or the divided region is displayed in a dashed line.

Finally the display unit 127 displays the graph 211 indicated on bottom left of the FIG. 8 as one row of divided regions R(3,1), R(3,2) R(3,3) and R(3,4). On the graph 211 indicated on bottom left of FIG. 8, the vertical axis indicates the movement distance (mm) and the horizontal axis indicates the time. The amount of movement in the vertical direction of divided regions R(3,1), R(3,2), R(3,3) and R(3,4) is indicated in the graph at each duration. In the graph, the lines indicated as g(3,1), g(3,2), g(3,3) and g(3,4) correspond to divided regions R(3,1), R(3,2), R(3,3) and R(3,4). Although it is not indicated in FIG. 8, the amount of movement of the divided regions DR in the horizontal direction can be displayed.

Based on these graphs, although the movement of the divided region R(3,4), which is equivalent to the surface of the plaque PQ, is small even after a certain duration, the movement of tissue in the divided region R(3,3) inside of the plaque PQ is considered to be large. Therefore, since the movement inside the tissue can be identified, the plaque characteristic can be more accurately determined than only by tracing the surface of a plaque.

At this point, among the divided regions DR, when the operator wants to observe one row of the divided regions R(1,3), R(2,3), R(3,3), R(4,3), R(5,3) R(6,3) and R(7,3), the operator selects one row of the divided regions R(1,3) to R(7,3) using the mouse pointer MP. One row of the selected divided regions DR is indicated in the different color or the frame of the divided region is indicated in the dashed line.

The display unit 127 displays the graph 213 indicated on the right of the FIG. 8 as one row of divided regions R(1,3), R(2,3), R(3,3), R(4,3), R(5,3) R(6,3) and R(7,3). On the graph 213 indicated on the right, the vertical axis indicates the movement distance (mm) and the horizontal axis indicates the time. The amount of movement in the vertical direction of the divided regions R(1,3)-R(7,3) is indicated in the graph at each duration. In the graph, the lines indicated as g(1,3) to g(7,3) correspond to divided regions R(1,3) to R(7,3). Although it is not indicated in FIG. 8, the amount of movement of the divided regions DR in the horizontal direction can be displayed.

Based on these graphs, although the movement of the divided regions R(1,3), R(6,3) is small even after a certain duration, the movement of tissue in the divided regions R(3,3) and R(4,3) is considered to be large. Therefore, since the movement inside the tissue can be identified, the plaque characteristic can be more accurately determined than only by tracing the surface of a plaque.

<Example 2 of Displaying the Traced Result>

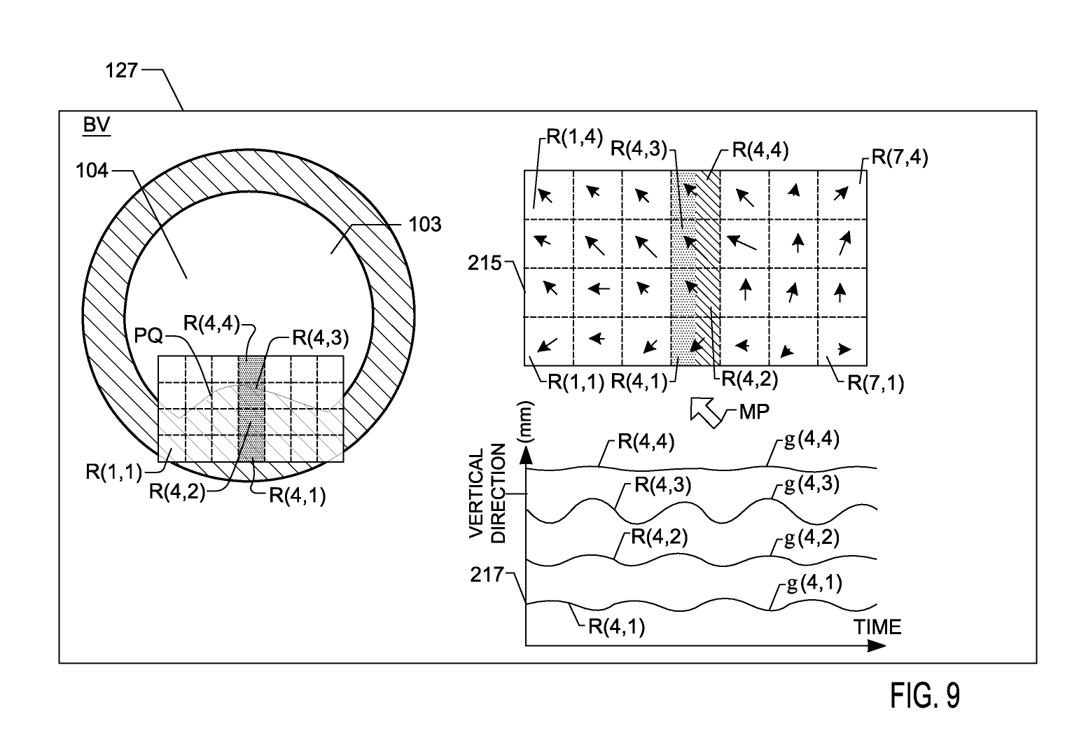

FIG. 9 shows a second example of the lined up graph of the traced result measured by the movement measurement unit 123 and the ultrasound image for displaying on the display unit 127. These graphs are displayed based on the total movement of a plurality of divided regions R(1,1)-R(7,4) indicated in the region of interest as shown in FIG. 5.

On left of the FIG. 9, the ultrasound image of the blood vessel wall in the short axis direction is displayed. On the ultrasound image, the divided regions DR of the ROI set by an operator are sequentially displayed. Also, on top right side of the FIG. 9, the divided regions DR of the ROI are displayed as chart 215. The divided regions DR indicated in chart 215 indicate the amount of the maximum movement during the predetermined duration as vectors.

At this point, among the divided regions DR, when the operator wants to observe one row of the divided regions R(4,1), R(4,2), R(4,3) and R(4,4), the operator selects one row of the divided regions R(4,1) to R(4,4) using the mouse pointer MP. One row of the selected divided regions DR is indicated in the different color or the frame of the divided regions is indicated in the dashed line. At the same time, the divided regions DR (left side of FIG. 9) of the ROI displayed on top of the ultrasound image is displayed in the different color for that one row.

The display unit 127 displays the graph 217 indicated on the bottom right of the FIG. 9 as one row of divided regions R(4,1) to R(4,4). On the graph 217 indicated on the bottom right, the vertical axis indicates the movement distance (mm) and the horizontal axis indicates the time. The amount of movement in the vertical direction of the divided regions R(4,1)-R(4,4) is indicated in the graph at each duration. Although it is not indicated in FIG. 9, the amount of movement of the divided regions DR in the horizontal direction can be displayed.

Based on lines g(4,1) to g(4,4), although the movement of the divided region R(4,4) is small even after a certain duration, the movement of tissue in the divided region R(4,3) is considered to be large. Therefore, since the movement inside the tissue can be identified, the plaque characteristic can be more accurately determined than only by tracing the surface of a plaque.

This embodiment indicates examples of the movement measurement unit 123 displaying the change in the amount of movement in the divided regions DR. The embodiment is not limited to examples described above. When the heartbeat or the blood pressure is measured, the movement measurement unit 123 can measure the stiffness parameter or the blood vessel wall diameter direction average elasticity coefficient.

Many widely different embodiments of the invention may be configured without departing from the spirit and the scope of the present invention. It should be understood that the present invention is not limited to the specific embodiments described in the specification, except as defined in the appended claims.

1. An ultrasound diagnostic apparatus comprising:
a transmitting and receiving unit configured to transmit an ultrasound wave to a target object in sequence and to receive the ultrasound wave as ultrasound data reflected from a certain region of the target object including a blood vessel in sequence;
a memory unit configured to store the received ultrasound data in sequence;
an image generation unit configured to generate an ultrasound image as a sectional image of the certain region based on the received ultrasound data;
a display unit configured to display the ultrasound image generated by the image generation unit;
a region of interest setting unit configured to set a region of interest including a plurality of divided regions in the displayed ultrasound image at a designated time, wherein the region of interest is generated from the ultrasound data stored in the memory unit;
a tracing unit configured to trace movement of tissue in the target object corresponding to the divided regions of the region of interest from the designated time to sequentially following thereafter; and
a movement measuring unit configured to measure a movement distance of the tissue at a predetermined time based on the movement of the tissue traced by the tracing unit.

2. The ultrasound diagnostic apparatus according to claim 1, wherein the region of interest setting unit is configured to set the region of interest as a square with square-shaped divided regions aligned in vertical and horizontal directions.

3. The ultrasound diagnostic apparatus according to claim 1, wherein the region of interest setting unit is configured to set the region of interest as a circular-shaped, elliptically shaped, fan-shaped or toric-shaped region including fan-shaped divided regions that are aligned in radiated and circular directions.

4. The ultrasound diagnostic apparatus according to claim 1, wherein the region of interest setting unit is configured to change a size of each divided region of the region of interest to a designated size.

5. The ultrasound diagnostic apparatus according to claim 2, wherein the region of interest setting unit is configured to change a size of each divided regions region of the region of interest to a designated size.

6. The ultrasound diagnostic apparatus according to claim 3, wherein the region of interest setting unit is configured to change a size of each divided region of the region of interest to a designated size.

7. The ultrasound diagnostic apparatus according to claim 1, wherein the tracing unit is configured to trace the movement of the tissue in the target object using an optical flow method between the ultrasound images.

8. The ultrasound diagnostic apparatus according to claim 2, wherein the tracing unit is configured to trace the movement of the tissue in the target object using an optical flow method between the ultrasound images.

9. The ultrasound diagnostic apparatus according to claim 3, wherein the tracing unit is configured to trace the movement of the tissue in the target object using an optical flow method between the ultrasound images.

10. The ultrasound diagnostic apparatus according to claim 7, wherein the optical flow method includes a gradient method using a spatial brightness gradient.

11. The ultrasound diagnostic apparatus according to claim 8, wherein the optical flow method includes a gradient method using a spatial brightness gradient.

12. The ultrasound diagnostic apparatus according to claim 9, wherein the optical flow method includes a gradient method using a spatial brightness gradient.

13. The ultrasound diagnostic apparatus according to claim 1, wherein the tracing unit is configured to determine that all of the region of interest is moved and to display the moved region of interest using the display unit when an amount of movement of each divided region is identical and each divided region moves in an identical direction.

14. The ultrasound diagnostic apparatus according to claim 1, wherein the region of interest includes a plaque formed on an inner wall of the blood vessel.

15. The ultrasound diagnostic apparatus according to claim 2, wherein the region of interest includes a plaque formed on an inner wall of the blood vessel.

16. The ultrasound diagnostic apparatus according to claim 3, wherein the region of interest includes a plaque formed on an inner wall of the blood vessel.

17. The ultrasound diagnostic apparatus according to claim 4, wherein the region of interest includes a plaque formed on an inner wall of the blood vessel.

18. The ultrasound diagnostic apparatus according to claim 7, wherein the region of interest includes a plaque formed on an inner wall of the blood vessel.

19. The ultrasound diagnostic apparatus according to claim 10, wherein the region of interest includes a plaque formed on an inner wall of the blood vessel.

20. A method for tracing movement tissue comprising:
transmitting an ultrasound wave to a target object in sequence;
receiving the ultrasound wave as ultrasound data reflected from a certain region of the target object including a blood vessel in sequence;
storing the received ultrasound data in sequence;
generating an ultrasound image as a sectional image of the certain region based on the received ultrasound data;
displaying the ultrasound image;
setting a region of interest including a plurality of divided regions in the displayed ultrasound image;
tracing movement of tissue in the target object corresponding to the divided regions of the region of interest from a designated time to sequentially following thereafter; and
measuring a movement distance of the tissue at a predetermined time based on the traced movement of tissue.
